(19) United States
(12) Patent Application Publication
(10) Pub. No.: US 2025/0066897 A1
(43) Pub. Date: Feb. 27, 2025

(54) HIGH-STRENGTH AND HIGH-TOUGHNESS ZN-MG-CA-SR ALLOY PREPARATION METHOD AND APPLICATION THEREOF

(71) Applicants: XI'AN JIAOTONG UNIVERSITY, Xi'an (CN); SHAANXI ZINC INDUSTRY CO., LTD., Shangluo (CN)

(72) Inventors: Shengqiang MA, Xi'an (CN); Ping LV, Xi'an (CN); Yusheng LIU, Xi'an (CN); Jiandong XING, Xi'an (CN); Jiankang ZHANG, Shangluo (CN); Jing XU, Shangluo (CN); Yaru XIA, Xi'an (CN); Maojiang YE, Xi'an (CN); Gaofeng MA, Shangluo (CN)

(21) Appl. No.: 18/941,180

(22) Filed: Nov. 8, 2024

(30) Foreign Application Priority Data
Jan. 12, 2024 (CN) 202410052146.4

(51) Int. Cl. C22F 1/16; C22C 18/00

(52) U.S. Cl. CPC C22F 1/165; C22C 18/00

(57) ABSTRACT

A method for preparing a Zn—Mg—Ca—Sr alloy, in which an as-cast Zn—Mg—Ca—Sr alloy is subjected to homogenization, and a boron nitride lubricant is sprayed on a surface of the as-cast Zn—Mg—Ca—Sr alloy and an inner cavity surface of a die. The as-cast Zn—Mg—Ca—Sr alloy is put into the die, and subjected to heating and 8-pass reciprocating extrusion to obtain the desired Zn—Mg—Ca—Sr alloy with high strength and high toughness, where the extrusion speed is stagewise controlled to realize stagewise variable-extrusion speed reciprocating extrusion. This application also provides a biodegradable medical implant material including a Zn—Mg—Ca—Sr alloy prepared by such method.

### CROSS-REFERENCE TO RELATED APPLICATIONS

This application claims the benefit of priority from Chinese Patent Application No. 202410052146.4, filed on Jan. 12, 2024. The content of the aforementioned application, including any intervening amendments thereto, is incorporated herein by reference in its entirety.

### TECHNICAL FIELD

This application relates to biodegradable medical implant materials, and more specifically to a high-strength and high-toughness Zn—Mg—Ca—Sr alloy, and a preparation method and an application thereof.

### BACKGROUND

With the advancement of medical technology, medical implants play an indispensable role in treating many diseases. However, conventional implants such as stainless steel, cobalt alloys, titanium alloys, and polymers have deficiencies, such as the need for secondary surgery for removal of the implants, and insufficient support strength. Those deficiencies negatively affect the physical and mental conditions of patients. Luckily, researches on biodegradable metal materials can greatly improve the above deficiencies. Among the intensively studied biodegradable metals, i.e., Fe, Zn, and Mg, the standard electrode potential (−0.76 V) of pure Zn is between that of Mg (−2.37 V) and that of Fe (−0.44 V). The degradation rate of pure Zn is more suitable to be used as a biodegradable implantable material for the human body, and its degradation products can be absorbed by the human body or metabolized. Hence, zinc is considered to be a more promising biodegradable material. However, the as-cast pure zinc has low mechanical properties, with a tensile strength of merely 33.6 MPa and an elongation of 1.2%, which limits its application as a biodegradable implant material. The alloying with different elements can improve the strength of zinc while also changing the corrosion rate of zinc.

Considering the corrosion, mechanical properties, and biocompatibility of alloying elements to zinc, the most widely studied alloying elements include Mg, Li, Cu, Ca, Sr, and Mn. Particularly, the mechanical properties of Zn—Mg, Zn—Li, and Zn—Cu alloys show the most significant enhancement while satisfying the baseline of clinical needs. Among those alloying elements, Mg, Ca, and Sr, as essential nutrients to the human body, are preferred for alloying with zinc. However, alloying usually fails to improve the ductility of zinc alloys, and generates a limited strength enhancement, with the strength not exceeding 200 MPa and the elongation of lower than 5% under the cast conditions. In some cases, alloying may significantly decrease the ductility.

Therefore, one of the most effective ways to simultaneously increase the strength and ductility of alloys is fine-grain toughening. Severe plastic deformation (SPD) technology can achieve grain refinement on the nanometer scale by introducing severe plastic strains into the metal, such as equal channel angular pressing, high-pressure torsion, reciprocating extrusion, and stacked rolling technology. It has been proved that sub-micron or nano-sized grains can be produced during the processing and formation of alloys. Among them, the reciprocating extrusion process can effectively eliminate various defects in the original materials, improve the distribution and shape of the reinforcement to form a uniform equiaxial fine grain microstructure, and facilitate continuously repeated deformation. With the increase in the number of passes of the reciprocating extrusion, the grains in the alloy are gradually refined. As the deformation resistance of the alloy gradually increases, if the multi-pass extrusion continuously undergoes under a constant extrusion speed, the flow stress of the alloy will be larger, the deformation through the concave die will be more difficult, and the damage to the concave die due to the subsequent deformation will also be aggravated. The combined die has a flexible and variable structure, but it also has deficiencies, such as an increase in die contact surface, and inevitable die clearance. At this time, with the increase in the number of passes of the reciprocating extrusion, it will inevitably result in the accumulation and extrusion of the material along the die contact surface, forming surface defects on the extrusion piece (such as flying edge, burr, and cracks).

Furthermore, the severe accumulation of the extrusion material also causes damage to the die, resulting in the difficulty of reciprocating extrusion and formation, severe friction and internal consumption, and significant increase in forming force, thereby greatly reducing the yield of finished products and the service life of the die, and even rendering the die scrapped. The research on reciprocating extrusion of magnesium alloys (AZ31 and WE43) shows that the temperature distribution during deformation is more inhomogeneous with the increase in the extrusion speed. This affects the homogeneity of the deformation microstructures of the material. Therefore, during the reciprocating extrusion process, with the increase in the number of extruding passes, the material microstructures will be denser and finer. However, if the extrusion continuously undergoes under a constant extrusion speed to obtain a greater cumulative strain of the material, the extrusion pressure will be continuously increased in the subsequent reciprocating extrusion process, and the extrusion process will become more and more difficult, which may increase the forming difficulty of reciprocating extrusion and have risks of damage to the die.

### SUMMARY

In view of this, objectives of the present disclosure are to provide a high-strength Zn—Mg—Ca—Sr alloy, a preparation method and an application thereof to overcome the deficiencies in the prior art, such as the formation difficulty of biodegradable zinc alloys in multi-pass reciprocal extrusion, poor uniformity of reciprocal extrusion, the difficulty in controlling the size of recrystallization grains, the low yield rate of the extruded parts, and poor strength and toughness of the as-extruded parts and products.

The technical solutions of the present disclosure are described below.

In a first aspect, this application provides a method for preparing a Zn—Mg—Ca—Sr alloy, comprising:
subjecting an as-cast Zn—Mg—Ca—Sr alloy to homogenization;
spraying a boron nitride lubricant on a surface of the as-cast Zn—Mg—Ca—Sr alloy and a surface of an inner cavity of a die; and
putting the as-cast Zn—Mg—Ca—Sr alloy into the die followed by heating and 8-pass reciprocating extrusion using a hydraulic press to obtain the Zn—Mg—Ca—Sr alloy;
wherein an extrusion speed of the 8-pass reciprocating extrusion is stagewise controlled to realize stagewise variable-extrusion speed reciprocating extrusion.

In an embodiment, the 8-pass reciprocating extrusion is performed through steps of:
controlling the extrusion speed to be 0.065-0.080 mm/s in the 1^{st}-3^{rd} passes;
controlling the extrusion speed to be 0.0275-0.040 mm/s in the 4^{th}-6^{th} passes; and
controlling the extrusion speed to be 0.008-0.015 mm/s in the 7^{th}-8^{th} passes.

In an embodiment, a pressure applied by the hydraulic press is 80-90 T.

In an embodiment, in the 8-pass reciprocating extrusion, after completing each pass of the reciprocating extrusion, the die is turned by 180° and kept under a heat preservation condition for 10-20 min.

In an embodiment, the homogenization is performed at 200-220° C. for 2.5-3.5 h.

In an embodiment, a thickness of the boron nitride lubricant is 15-30 μm.

In an embodiment, the heating is performed at 250-280° C. for 0.5-1 h.

In a second aspect, this application provides an Zn—Mg—Ca—Sr alloy prepared by the above-mentioned method, comprising 0.95-1.30% by weight of Mg, 0.15-0.20% by weight of Ca, 0.08-0.12% by weight of Sr, and Zn for balance.

In an embodiment, the Zn—Mg—Ca—Sr alloy has a strength of 300-350 MPa and an elongation of 10-15%.

In a third aspect, this application provides a biodegradable human tissue implant material, comprising the aforementioned Zn—Mg—Ca—Sr alloy.

Compared with the prior art, the present application has at least the following beneficial effects.

In the method provided herein, the as-cast Zn—Mg—Ca—Sr alloy is subjected to stagewise variable-speed reciprocating extrusion, where the extrusion speed is relatively large in the initial stage, and then decreases in the subsequent stages, such that in different passes, the alloy is extruded at different extrusion speeds. Through the multi-pass reciprocating extrusion, the recrystallization structure is stagewise refined, and gradually becomes flowable with the increase in the number of passes. The descending gradient distribution of the extrusion speed contributes to the effective and complete deformation of the hard-deformed biodegradable zinc alloys, and also plays a role in protecting the die for the multi-pass reciprocating extrusion, thereby improving the yield and extending the service life of the die. By using the multi-pass variable-speed reciprocating extrusion, where the extrusion speed decreases stepwise during the gradual densification of the billet, the alloy undergoes dynamic recrystallization to form a large number of undistorted isometric crystals. The recrystallized grains are significantly refined to the micron level or below, with an overall structure distributed linearly and parallel to the extrusion direction. In this case, the original lamellar eutectic structure disappears and is transformed into a second phase with solid solution and intragranular dispersive distribution. The second phase (reinforcing phase) is broken and distributed along the extrusion direction, and the sharp angles become round and passivated, which effectively improves the strength and elongation of the zinc alloy, significantly refines the grain size of the Zn—Mg—Ca—Sr alloy, and improves the size, shape, and distribution of the reinforcing phase (namely, offering dual effects of fine-grain strengthening and second-phase strengthening). This application provides a new research strategy for further development of biodegradable medical materials.

In an embodiment, the stagewise variable-speed reciprocating extrusion is carried out, such that the extrusion speed varied in different passes, thereby realizing the descending gradient distribution of the extrusion speed. In this way, the extrusion speed is controlled to be 0.065-0.080 mm/s in the 1^{st}-3^{rd} passes, 0.0275-0.040 mm/s in the 4^{th}-6^{th} passes, and 0.008-0.015 mm/s in the 7^{th}-8^{th} passes. The extrusion yield is increased by 96%, and the service life of the die is increased by 150%.

In an embodiment, in the 8-pass reciprocating extrusion, after completing each pass of the reciprocating extrusion, the die is turned by 180° and kept under a heat preservation condition for 10-20 min. The multi-pass reciprocating extrusion can fully deform the alloy, effectively improve the grain refinement effect, and improve the shape, size and distribution of the reinforcing phase.

In an embodiment, the homogenization treatment can eliminate the composition segregation in the as-cast Zn—Mg—Ca—Sr alloy, and improve the uniformity of the alloy.

In an embodiment, before the as-cast Zn—Mg—Ca—Sr alloy is placed into the die, a boron nitride lubricant with a thickness of 15-30 μm is sprayed on a surface of the as-cast Zn—Mg—Ca—Sr alloy and a surface of an inner cavity of the die. This can prevent the bonding and thermal adhesion of billet alloy and die mold produced during the deformation process and reduce the friction of reciprocating extrusion. Compared with the situation that no lubricant is sprayed on the surface of the alloy sample and the surface of the inner cavity of the die, the lubrication protection effect in the present disclosure is remarkable to make the reciprocating extrusion process smooth and easy.

In an embodiment, zinc has a hexagonal close-packed (HCP) structure. When heated to 250-280° C. and kept at such temperature for 0.5-1 h, the ductility of zinc alloy can be effectively improved, which can prevent the material cracking during the reciprocating extrusion process. The melting point of the as-cast Zn—Mg—Ca—Sr alloy is about 362° C. The extrusion is performed at 250-280° C., such that the dynamic recrystallization of a-Zn occurs during the extrusion to form undistorted equiaxed crystals, thereby improving the strength and ductility of the alloy.

According to the Zn—Mg, Zn—Ca and Zn—Sr phase diagrams, α-Zn dendrites, lamellar Zn+Mg₂Zn₁₁ eutectic structure and massive (Ca,Sr)Zn₁₃ intermetallic compounds are formed during the solidification process of the Zn—Mg—Ca—Sr alloy. When Mg content is set to 0.95-1.30%, the generated Zn+Mg₂Zn₁₁ eutectic structure can significantly improve the strength and hardness of the alloy. Moreover, the addition of Ca and Sr can play the effect of metamorphic treatment to refine the grain, and further improve the strength of the alloy.

To sum up, in this application, the alloy composition, the extrusion pressure, the extrusion temperature, the extrusion speed and the type of coating are desirable, and the multi-pass variable-speed reciprocating extrusion is utilized. After conducting the 8-pass reciprocating extrusion, the grain size is reduced by 300%, and the recrystallization grain is refined to 0.5 μm, compared with the grains produced under a constant extrusion speed. Moreover, the recrystallization grains are round and uniform, the shape of the strengthening phase is round and uniform in distribution, the tensile strength of the zinc alloy is increased to more than 340 MPa, and the elongation of the zinc alloy is as high as 12%.

The technical solutions of the present disclosure will be further described though the following drawings and embodiments.

### BRIEF DESCRIPTION OF THE DRAWINGS

FIG. 1 is a schematic diagram of a reciprocating extrusion tooling according to an embodiment of the present disclosure;

FIG. 2 shows an extrusion speed distribution in various passes during the variable-speed reciprocating extrusion according to an embodiment of the present disclosure;

FIGS. 3a-3c show morphological images of a Zn—Mg—Ca—Sr alloy at different stages according to an embodiment of the present disclosure, where (a) as-cast; (b) as-extruded from example 1; (c) as-extruded from example 3;

FIGS. 4a-4b respectively show a grain size distribution and a grain boundary distribution of the Zn—Mg—Ca—Sr alloy under four-pass variable-speed reciprocating extrusion according to an embodiment of the present disclosure; and

FIG. 5 is a tensile curve of the Zn—Mg—Ca—Sr alloy at room temperature after the four-pass variable-speed reciprocating extrusion according to an embodiment of the present disclosure.

In the drawings, 1, die base; 2, spacer block; 3, buffer spring; 4, screw; 5, extrusion cylinder; 6, extrusion rod; 7, die; and 8, fixing clamp.

### DETAILED DESCRIPTION OF EMBODIMENTS

The technical solutions in the embodiments of the present disclosure will be described clearly and completely below. Obviously, the described embodiments are only a part of the embodiments of the present disclosure. Based on these embodiments, all other embodiments obtained by one of ordinary skill in the art without making creative labor shall fall within the scope of protection of the present disclosure.

In the present disclosure, unless otherwise specified, all embodiments and preferred embodiments mentioned herein can be combined to form a new technical scheme.

In the present disclosure, unless otherwise specified, all technical features and preferred features mentioned herein can be combined to form a new technical solution.

In the present disclosure, unless otherwise specified, the percentage (%) or the part refers to the weight percent or the weight part relative to the compositions.

In the present disclosure, unless otherwise specified, components or optimization thereof involved herein can be combined to form a new technical solution.

In the present disclosure, unless otherwise specified, the numerical range “a-b” is an abbreviation referring to any real combination from a to b, where a and b are real numbers. For example, the numerical range “6-22” indicates that all real numbers from 6 to 22 are listed herein, which is only an abbreviation of the combinations of these values.

The “scope” disclosed herein consists of a lower limit and an upper limit, which may be one or more lower limits, and one or more upper limits, respectively.

In the present disclosure, the term “and/or” as used herein refers to any combination and all possible combinations of one or more of the items listed in association and includes these combinations.

In the present disclosure, unless otherwise indicated, the individual reaction or operation steps may be performed in sequence. Preferably, the steps in the method provided herein are performed sequentially.

Unless otherwise indicated, professional and scientific terms used herein have the same meaning as those familiar to one of ordinary skill in the art. In addition, any method or material similar or homogeneous to what is disclosed herein may also be applied in the present disclosure.

The present disclosure provides a method for preparing an Zn—Mg—Ca—Sr alloy and an application thereof. The as-cast Zn—Mg—Ca—Sr alloy is subjected to stagewise variable-speed reciprocating extrusion, where the extrusion speed decreases stagewise with a gradient changes, i.e., utilizing a first-rapid-then-slow extrusion speed distribution way during whole reciprocating extrusion process, so as to obtain ultra-fine reciprocating extruded Zn—Mg—Ca—Sr alloy with a high strength and a good elongation. As can be seen from the structure of the as-cast Zn—Mg—Ca—Sr alloy, the a-Zn dendrites matrix, a small amount of the lamellar Zn+Mg₂Zn₁₁ eutectic structure and a small amount of bulk (Ca,Sr)Zn₁₃ intermetallic compounds exist in the as-cast Zn—Mg—Ca—Sr alloy. After the reciprocating extrusion, the a-Zn phase matrix undergoes dynamic recrystallization. As a result, the grains are significantly refined with a streamlined distribution along a direction of the extrusion; the lamellar eutectic structure disappears, and solid solution phase or dispersive fine precipitation phase appears; and the bulk reinforcing phase (Ca, Sr)Zn₁₃ is broken with sharp corners being rounded. Through the combination of the fine-grain strengthening and the second-phase strengthening, the grains of the alloy are significantly refined to the micron level or below, and the average tensile strength of the extruded-state alloy is increased to 340 MPa, and the elongation of the extruded-state alloy is increased to 12%.

In the method provided herein, the as-cast Zn—Mg—Ca—Sr alloy is used as the raw material and subjected to stagewise variable-speed reciprocating extrusion, where the extrusion speed is relatively large in the initial stage, and then decreases in the subsequent stages, that means, with a gradient change, a first-rapid-then-slow extrusion speed distribution way during whole reciprocating extrusion process is put forward to achieve the microstructural refinement. Through the multi-pass reciprocating extrusion with a first-rapid-then-slow extrusion speed distribution way, the recrystallization microstructure is remarkably refined, and gradually becomes flowable and controllable under a lower flow stress of the alloy with the increase in the number of passes. By stagewise controlling the extrusion speed of reciprocating extrusion, the 8-pass stagewise variable-speed reciprocating extrusion process is realized, and the extrusion speed decreases in gradient with the step-by-step refinement and densification as well as the gradual reduced flow stress deformation of the billet. The method includes the following steps.

(S1) An as-cast Zn—Mg—Ca—Sr alloy is subjected to homogenization at 200-220° C. for 2.5-3.5 h and turning machining to reach a shape fitting an inner cavity of a die of a reciprocating extrusion tooling.

(S2) Both of the as-cast Zn—Mg—Ca—Sr alloy obtained in step (S1) and the inner cavity surface of the die are sprayed with a boron nitride lubricant with a thickness of 15-30 μm, and then the as-cast Zn—Mg—Ca—Sr alloy above treated is put into the die. An upper barrel, a lower barrel, an extrusion rod, a heating coil, and a fixing clamp are assembled, and the die is connected with a reciprocating extrusion bracket. A hydraulic press is used to pre-compact the die to eliminate the gap between the billet and the die.

(S3) The heating coil is connected with a thermocouple and a temperature control device, and wrapped with a fire-resistant cotton. The heating temperature is set to 250-280° C., and the heating coil is turned on for heating the die.

(S4) The billet is kept at 250-280° C. for 0.5-1 h, and the hydraulic press is turned on to complete a first pass of extrusion at an extrusion speed of 0.08 mm/s and a pressure of 80-90 T.

(S5) The extrusion speed is controlled to be 0.065-0.080 mm/s at a first stage (1^{st}-3^{rd} passes) of the extrusion, where the extrusion speed is relatively large, facilitating the recrystallization refinement.

The die is turned by 180°, and heated to a specified temperature and kept at the specified temperature for 10-20 min. After that, the hydraulic press is turned on to complete a second pass of extrusion at an extrusion speed of 0.070 mm/s, then the die is turned over, kept at the specified temperature for 15 min and extruded at an extrusion speed of 0.065 mm/s to complete a third-pass extrusion.

(S6) The extrusion speed is controlled to be 0.0275-0.040 mm/s at a second stage (4^{th}-6^{th} passes) of the extrusion, where the extrusion speed is medium, facilitating the recrystallization refinement and the grain homogenization.

The extrusion in the 4^{th}-6^{th} passes is performed respectively at a speed of 0.040 mm/s, 0.030 mm/s, 0.0275 mm/s according to operations in step (S5).

(S7) The extrusion speed is controlled to be 0.008-0.015 mm/s at a third stage (7^{th}-8^{th} passes) of the extrusion, where the extrusion speed is slow, facilitating the recrystallization super-refinement and further deformation uniformity.

Step (S5) is repeated, and the extrusion speeds at the subsequent 7^{th}-8^{th} passes of extrusion are 0.015 mm/s and 0.008 mm/s, respectively.

After completing each pass of extrusion, the die is turned by 180° and kept at the certain holding temperature for 10-20 min to complete the next pass of reciprocating extrusion. The temperature and the pressure at each pass of extrusion are kept the same, that is, the process of each pass of the reciprocating extrusion is completely equivalent except a variable gradient-distributed extrusion speed (i.e. variable-speed) as each stage.

Preferably, the die is insulated with the fire-resistant cotton and insulation felt during heating and extrusion.

(S8) The die is disassembled, and placed on the sleeve. The hydraulic press is turned on to press the as-extruded billet out, i.e., the extruded-state Zn—Mg—Ca—Sr alloy billet is obtained through the stagewise variable-speed reciprocating extrusion.

The 8-pass stagewise variable-speed reciprocating extrusion is performed to obtain a Zn—Mg—Ca—Sr alloy with a high strength and a high toughness.

The Zn—Mg—Ca—Sr alloy provided herein includes 0.95-1.30% by weight of Mg, 0.15-0.20% by weight of Ca, 0.08-0.12% by weight of Sr, and Zn for balance.

In the method provided herein, the alloy composition, the extrusion pressure, the extrusion temperature, the extrusion speed and the type of coating are desirable. In addition, the multi-pass variable-speed reciprocating extrusion is utilized. After conducting the 8-pass reciprocating extrusion, the grain size is reduced by 300%, and the recrystallization grain is refined to 0.5 μm, compared with the grains produced under a constant extrusion speed. Moreover, the recrystallization grains are round and uniform, the shape of the strengthening phase is round and uniform in distribution, the tensile strength of the zinc alloy is up to 300-350 MPa, and the elongation of the zinc alloy is 10-15%. Therefore, the alloy provided herein has a wide range of applications in biodegradable human tissue implant materials (such as orthopedic implant medical devices and cardiovascular stents).

The zinc alloy prepared using the stagewise variable-speed control reciprocating extrusion of the present disclosure has a high strength and a high toughness. During the reciprocating extrusion, the extrusion speed is relatively large in the initial stage, and then decreases in the subsequent stages, such that the billet can fully flow, which can improve the yield rate and the forming efficiency of the bio-zinc alloy, and prolong the service life of the reciprocating extrusion die, so as to accelerate the application of zinc alloy in the clinical medical materials.

To make the purpose, technical solution ns and advantages of the embodiments of the present disclosure clearer, the technical solutions in the embodiments of the present disclosure will be described clearly and completely below with reference to the accompanying drawings. Obviously, described herein are merely some embodiments of the present disclosure, instead of all embodiments. The components of embodiments described herein and shown in the accompanying drawings can be arranged and designed in a variety of different configurations. Accordingly, the following detailed description of embodiments of the present disclosure provided in the accompanying drawings is not intended to limit the scope of the present disclosure, but rather represents selected embodiments of the present disclosure. Based on the embodiments in the present disclosure, all other embodiments obtained by one of ordinary skill in the art without creative effort shall fall within the scope of the present disclosure.

### Example 1

An as-cast Zn—Mg—Ca—Sr alloy was subjected to homogenization treatment at 210° C. for 3.0 h, and turning machining to reach a shape fitting an inner cavity of a die of a reciprocating extrusion tooling. The as-cast Zn—Mg—Ca—Sr alloy and the inner cavity of the die were sprayed with a boron nitride lubricant with a thickness of 30 μm, and then the as-cast Zn—Mg—Ca—Sr alloy was put into the die. The reciprocating extrusion tooling was assembled, and the die was heated to 260° C. and kept at 260° C. for 1 h. A hydraulic press was turned on to complete a first pass of extrusion at an extrusion speed of 0.08 mm/s and a pressure of 82 T. After that, the die was turned by 180° and kept at 260° C. for 20 min. The hydraulic press was turned on to successively complete 2^{nd}-8^{th} passes of extrusion at an extrusion speed of 0.070 mm/s, 0.065 mm/s, 0.040 mm/s, 0.030 mm/s, 0.0275 mm/s, 0.015 mm/s, and 0.008 mm/s, respectively. After that, the reciprocating extrusion tooling was disassembled, and the Zn—Mg—Ca—Sr alloy with high strength and high toughness was obtained after demolding.

### Example 2

An as-cast Zn—Mg—Ca—Sr alloy was used as a raw material, subjected to a homogenization treatment at 200° C. for 3.5 h, and turning machining to reach a shape fitting an inner cavity of a die of a reciprocating extrusion tooling. The as-cast Zn—Mg—Ca—Sr alloy and the inner cavity of the die were sprayed with a boron nitride lubricant with a thickness of 20 μm, and then the as-cast Zn—Mg—Ca—Sr alloy was put into the die. The reciprocating extrusion tooling was assembled, and the die was heated to 280° C. and kept at 280° C. for 30 min. A hydraulic press was turned on to complete a first pass of extrusion at an extrusion speed of 0.08 mm/s and a pressure of 80 T. After that, the die was turned by 180° and kept at 280° C. for 15 min. The hydraulic press was turned on to successively complete 2^{nd}-8^{th} passes of extrusion at an extrusion speed of 0.070 mm/s, 0.065 mm/s, 0.040 mm/s, 0.030 mm/s, 0.0275 mm/s, 0.015 mm/s, and 0.008 mm/s, respectively. After that, the reciprocating extrusion tooling was disassembled, and the Zn—Mg—Ca—Sr alloy with high strength and high toughness was obtained after demolding.

### Example 3

An as-cast Zn—Mg—Ca—Sr alloy was used as a raw material, subjected to a homogenization treatment at 220° C. for 2.5 h, and turning machining to reach a shape fitting an inner cavity of a die of a reciprocating extrusion tooling. The as-cast Zn—Mg—Ca—Sr alloy and the inner cavity of the die were sprayed with a boron nitride lubricant with a thickness of 15 μm, and then the as-cast Zn—Mg—Ca—Sr alloy was put into the die. The reciprocating extrusion tooling was assembled, and the die was heated to 250° C. and kept at 250° C. for 45 min. A hydraulic press was turned on to complete a first pass of extrusion at an extrusion speed of 0.08 mm/s and a pressure of 90 T. After that, the die was turned by 180° and kept at 250° C. for 10 min. The hydraulic press was turned on to successively complete 2^{nd}-8^{th} passes of extrusion at an extrusion speed of 0.070 mm/s, 0.065 mm/s, 0.040 mm/s, 0.030 mm/s, 0.0275 mm/s, 0.015 mm/s, and 0.008 mm/s, respectively. After that, the reciprocating extrusion tooling was disassembled, and the Zn—Mg—Ca—Sr alloy with high strength and high toughness was obtained after demolding.

### Example 4

An as-cast Zn—Mg—Ca—Sr alloy was used as a raw material, subjected to a homogenization treatment at 215° C. for 2.5 h, and turning machining to reach a shape fitting an inner cavity of a die of a reciprocating extrusion tooling. The as-cast Zn—Mg—Ca—Sr alloy and the inner cavity of the die were sprayed with a boron nitride lubricant with a thickness of 20 μm, and then the as-cast Zn—Mg—Ca—Sr alloy was put into the die. The reciprocating extrusion tooling was assembled, and the die was heated to 275° C. and kept at 275° C. for 50 min. A hydraulic press was turned on to complete a first pass of extrusion at an extrusion speed of 0.08 mm/s and a pressure of 84 T. After that, the die was turned by 180° and kept at 275° C. for 15 min. The hydraulic press was turned on to successively complete 2^{nd}-8^{th} passes of extrusion at an extrusion speed of 0.070 mm/s, 0.065 mm/s, 0.040 mm/s, 0.030 mm/s, 0.0275 mm/s, 0.015 mm/s, and 0.008 mm/s, respectively. After that, the reciprocating extrusion tooling was disassembled, and the Zn—Mg—Ca—Sr alloy with high strength and high toughness was obtained after demolding.

### Example 5

An as-cast Zn—Mg—Ca—Sr alloy was used as a raw material, subjected to a homogenization treatment at 205° C. for 3.5 h, and turning machining to reach a shape fitting an inner cavity of a die of a reciprocating extrusion tooling. The as-cast Zn—Mg—Ca—Sr alloy and the inner cavity of the die were sprayed with a boron nitride lubricant with a thickness of 25 μm, and then the as-cast Zn—Mg—Ca—Sr alloy was put into the die. The reciprocating extrusion tooling was assembled, and the die was heated to 270° C. and kept at 270° C. for 35 min. A hydraulic press was turned on to complete a first pass of extrusion at an extrusion speed of 0.08 mm/s and a pressure of 86 T. After that, the die was turned by 180° and kept at 270° C. for 12 min. The hydraulic press was turned on to successively complete 2^{nd}-8^{th} passes of extrusion at an extrusion speed of 0.070 mm/s, 0.065 mm/s, 0.040 mm/s, 0.030 mm/s, 0.0275 mm/s, 0.015 mm/s, and 0.008 mm/s, respectively. After that, the reciprocating extrusion tooling was disassembled, and the Zn—Mg—Ca—Sr alloy with high strength and high toughness was obtained after demolding.

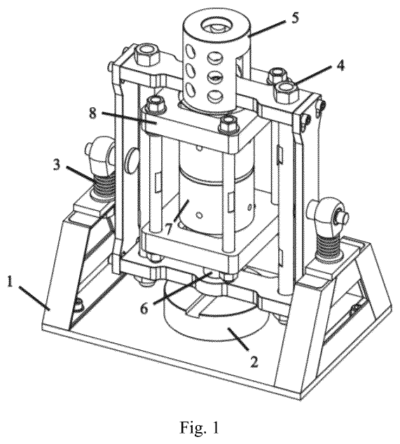

The reciprocating extrusion tooling was shown in FIG. 1, which included a die base 1, a spacer block 2, a buffer spring 3, a screw 4 for fixing a die holder, an extrusion cylinder 5, an extrusion rod 6, a die 7, and a fixing clamp 8. When performing the reciprocating extrusion, the billet was put into the die 7, and the die 7 was fixed by the fixing clamp 8. Subsequently, the extrusion rods 6 as two separating parts are placed from the ends of the mold (i.e. two cylinder dies of 7 as mold cavity and placing an internal die with a centre hole for extruded billet or blank through out into the middle position of the two cylinder dies of 7) and connected to the mold holder (i.e. connecting disc for fixing two die extrusion cylinders together to form a monolithic extrusion die mechanism) by means of screws. After completing the connection between the extrusion rods and the mold holder, the screw 4 is tightened, and then the backing block 2 at the die bottom and the extrusion cylinder 5 are placed to complete the subsequent extrusion process.

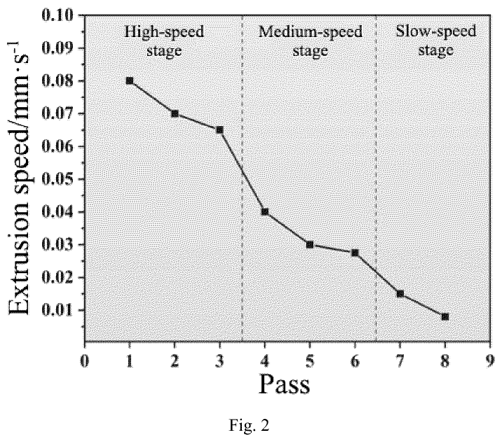

An extrusion speed distribution in various passes during the variable-speed reciprocating extrusion (i.e., a variable gradient-distributed extrusion speed vs. reciprocating extrusion pass) was shown in FIG. 2. As the number of passes increased, the deformation resistance within the material increased, and the flow stress required for deformation of the material decreased, and the cumulative strain in the billet dramatically increased. The extrusion speed was stepwise controlled, which avoided serious accumulation of the extruded blank in the deformation zone and non-uniform deformation, thereby smoothly obtaining the extruded blank with excellent surface quality with a comprehensive yield increased by 96%. Compared with the constant-speed extrusion, the variable-speed reciprocating extrusion had an extrusion efficiency increased by 65%, which also protected the die, so that the service life of the die increased by 150%. Comparisons between the constant-speed extrusion and the variable-speed reciprocating extrusion in terms of yield and die service life were shown in Table 1 below.

TABLE 1
Comparison between the constant-speed extrusion and the variable-
speed reciprocating extrusion in terms of yield and die service life

| | | | | | Increase in |
|---|---|---|---|---|---|
| | | | | | the service |
| | | | Continuous service | | life of the die |
| | | | life of the die | | compared |
| | Comprehensive | | under undamaged | | with the |
| | yield (%) | | conditions (month) | | constant-speed |
| | | Variable- | | Variable- | extrusion (%) |
| | | Speed | | Speed | Variable- |
| | Constant- | Recipro- | Constant- | Recipro- | Speed |
| Extrusion | Speed | cating | Speed | cating | Reciprocating |
| Passes | Extrusion | Extrusion | Extrusion | Extrusion | Extrusion |
| 2 | 92.0 | 98.0 | 20 | 22 | 10 |
| 4 | 72.4 | 95.1 | 9 | 15 | 60 |
| 8 | 47.8 | 93.5 | 2 | 5 | 150 |

The yield and the service life of the die were increased when adopting the stagewise variable speed control reciprocating extrusion for the following reasons. With the increase of extrusion passes, the internal microstructure of the material was gradually homogenized, and defects such as shrinkage and loosening (i.e., micro porosity by casting) gradually disappeared. Meanwhile, the matrix dendritic grains were subjected to refinement and spheroidization, and the resistance for continuous deformation of the alloy was also gradually increased. At this time, by increasing the extrusion pressure or reducing the extrusion speed can achieve subsequent reciprocating extrusion deformation of the alloy. Considering the forcing-saving formation, microstructural refinement and grain uniformity, smaller recrystallization grain control and die life extension to full exert the flow stress of high-temperature deformation of the material and recrystallized grain uniformity and homogeneity, the stagewise variable speed control reciprocating extrusion under the appropriate extrusion pressure was provided herein.

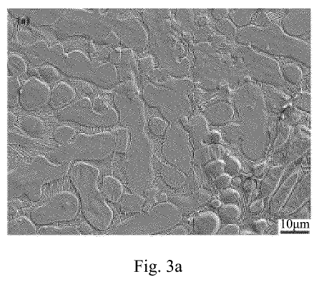

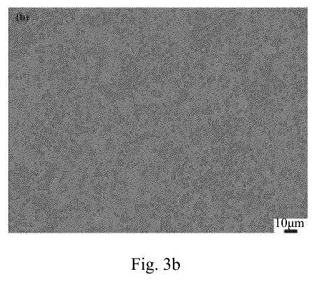

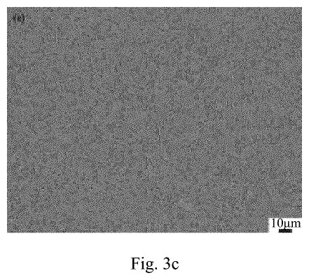

Microstructural images of a Zn—Mg—Ca—Sr alloy at different stages were shown in FIGS. 3a-3c. The as-cast Zn—Mg—Ca—Sr alloy included the a-Zn dendrites matrix, a small amount of the lamellar Zn+Mg₂Zn₁₁ eutectic structure and a small amount of bulk (Ca,Sr)Zn₁₃ intermetallic compounds. After the four passes of reciprocating extrusion at 250° C., the a-Zn phase matrix experienced dynamic recrystallization. As a result, the equiaxial recrystallized grains were significantly refined with a streamlined distribution along a direction of the extrusion; the lamellar eutectic structure disappeared, and solid solution or dispersive fine precipitation phases appeared; and the bulk reinforced phase (Ca,Sr)Zn₁₃ was broken with sharp corners being rounded. Compared with constant-speed reciprocating extrusion, the variable-speed reciprocating extrusion resulted in more complete deformation of the alloy and more uniform microstructural distribution.

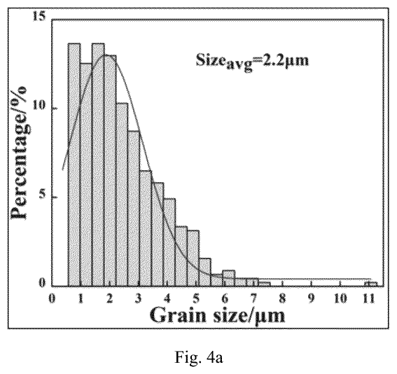

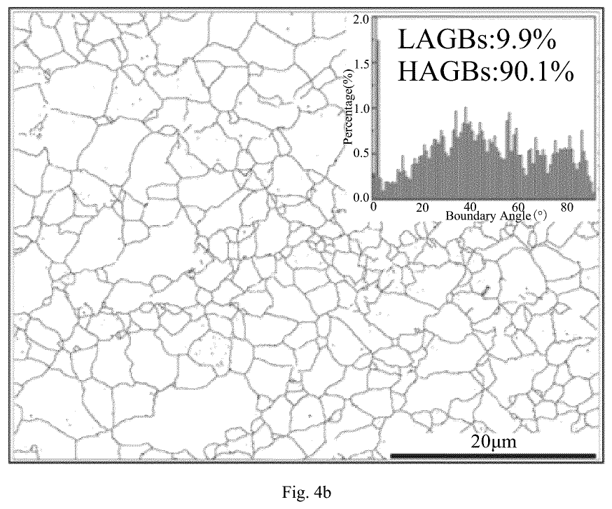

A grain size distribution and a grain boundary distribution of the Zn—Mg—Ca—Sr alloy under four-pass variable-speed reciprocating extrusion were shown in FIGS. 4a-4b, respectively. After four passes of reciprocating extrusion at 250° C., the alloy experienced dynamic recrystallization to form equiaxial crystals, and the grain size was refined to an average value of 2.2 μm, with the large-angle grain boundaries accounting for 90.1% and the small-angle grain boundaries accounting for 9.9%, indicating that the dynamic recrystallization occurred more fully and thoroughly.

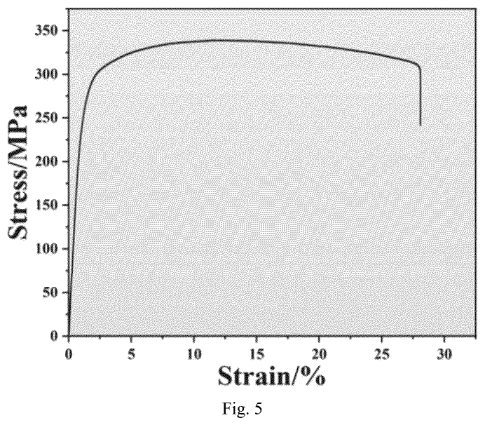

A tensile curve of the Zn—Mg—Ca—Sr alloy at room temperature after the four-pass variable-speed reciprocating extrusion was shown in FIG. 5. After four passes of reciprocating extrusion at 250° C., the tensile strength of the Zn—Mg—Ca—Sr alloy reached 340 MPa and the elongation reached 12%.

In conclusion, in the preparation method provided herein, through the stagewise variable-speed control reciprocating extrusion severe plastic deformation (SPD) technology, the composition segregation in the as-cast Zn—Zn—Mg—Ca—Sr alloy can be effectively eliminated, so that the alloy fully occurs in dynamic recrystallization, the recrystallized grains are significantly refined to sub-micron to form a uniform equiaxial fine grain microstructure. In addition, the shape of the reinforcing phase is rounded and uniformly distributed, the tensile strength of the zinc alloy is increased to 300-350 MPa, and the elongation of the zinc alloy is 10%-15%.

Finally, it should be noted that the above embodiments are only used to illustrate the technical solutions of the present disclosure, and are not intended to limit the present disclosure. Although the present disclosure has been described in detail with reference to the foregoing embodiments, one of ordinary skill in the art should understand that it is still possible to modify the technical solutions recorded in the foregoing embodiments, or to replace some or all of the technical features with equivalent ones. Those modifications or substitutions made without departing from the spirit of the present disclosure shall fall within the scope of the present disclosure defined by the appended claims.

What is claimed is:

1. A method for preparing a Zn—Mg—Ca—Sr alloy, comprising:
subjecting an as-cast Zn—Mg—Ca—Sr alloy to homogenization;
spraying a boron nitride lubricant on a surface of the as-cast Zn—Mg—Ca—Sr alloy and a surface of an inner cavity of a die; and
putting the as-cast Zn—Mg—Ca—Sr alloy into the die followed by heating and 8-pass reciprocating extrusion using a hydraulic press to obtain the Zn—Mg—Ca—Sr alloy;
wherein an extrusion speed of the 8-pass reciprocating extrusion is stagewise controlled to realize stagewise variable-extrusion speed reciprocating extrusion.

2. The method of claim 1, wherein the 8-pass reciprocating extrusion is performed through steps of:
controlling the extrusion speed to be 0.065-0.080 mm/s in 1^{st}-3^{rd} passes;
controlling the extrusion speed to be 0.0275-0.040 mm/s in 4^{th}-6^{th} passes; and
controlling the extrusion speed to be 0.008-0.015 mm/s in 7^{th}-8^{th} passes.

3. The method of claim 2, wherein a pressure applied by the hydraulic press is 80-90 T.

4. The method of claim 2, wherein in the 8-pass reciprocating extrusion, after completing each pass of extrusion, the die is turned by 180° and kept under a heat preservation condition for 10-20 min.

5. The method of claim 1, wherein the homogenization is performed at 200-220° C. for 2.5-3.5 h.

6. The method of claim 1, wherein a thickness of the boron nitride lubricant is 15-30 μm.

7. The method of claim 1, wherein the heating is performed at 250-280° C. for 0.5-1 h.

8. A Zn—Mg—Ca—Sr alloy prepared by the method of claim 1, wherein the Zn—Mg—Ca—Sr alloy comprises 0.95-1.30% by weight of Mg, 0.15-0.20% by weight of Ca, 0.08-0.12% by weight of Sr, and Zn for balance.

9. The Zn—Mg—Ca—Sr alloy of claim 8, wherein the Zn—Mg—Ca—Sr alloy has a strength of 300-350 MPa and an elongation of 10-15%.

10. A biodegradable medical implant material, comprising:
the Zn—Mg—Ca—Sr alloy of claim 8.
